(19) United States
(12) Patent Application Publication
(10) Pub. No.: US 2026/0283652 A1
(43) Pub. Date: Sep. 24, 2026

(54) SHANK AND TULIP ASSEMBLIES FOR SPINAL SURGERY

(71) Applicant: GLOBUS MEDICAL, INC., AUDUBON, PA (US)

(72) Inventors: Alex Balus, San Diego, CA (US); Matthew Ono, Nederland, CO (US); Ryan Woods, Broomf ield, CO (US); Eric Forman, San Diego, CA (US); Christian Yee-Yanagishita, San Diego, CA (US); Mario Villalpando, El Cajon, CA (US)

(21) Appl. No.: 19/082,311

(22) Filed: Mar. 18, 2025

(51) Int. Cl. A61B 17/70; A61B 17/86

(52) U.S. Cl. CPC A61B 17/7037; A61B 17/7032; A61B 17/8625

(57) ABSTRACT

Shank and tulip assemblies for spinal procedures include a fusion shank with features fostering bone fusion and a tulip component. Tulips may be configured so that shanks received therein are movable between a plurality of arcuate positions, making such tulips polyaxial. The fusion shank is used in conjunction with a tulip and, optionally, a companion implant.

### FIELD

This disclosure relates to spinal surgical systems in general, and to spinal fixation systems more particularly.

### BACKGROUND

Spinal fixation systems often make use of one or more shanks that serve as a bone anchor or anchors at one or more locations on the spine. Such bone anchors are often interconnected by one or more rods. Spinal procedures often make use of so-called tulips, which are connectors between a shank and an associated rod.

Certain spinal procedures, such as those involving the sacroiliac joint (SIJ), often make use of the posterior approach, which may be associated with a variety of trajectories, and include traditional S2-alar-iliac (S2AI), modified S2AI, traditional iliac, and modified iliac. In such procedures, it is often desirable for the fixation systems to include elements which foster fusion to bone.

### SUMMARY

In one implementation, a polyaxial tulip for connecting to a shank and a rod associated with a spinal procedure is provided. The tulip comprises a cylindrical body having a longitudinal axis, and defined by a pair of arcuate walls extending longitudinally between upper and lower ends of the tulip, the arcuate walls extending circumferentially through respective arcs to define a pair of slots in the cylindrical body, the slots configured to be able to selectively receive the rod therethrough. The cylindrical body having first and second openings defined at the lower and upper ends of the body, respectively; a split ring disposed in the first opening, the split ring having an inner diameter configured to secure a head of the shank when the shank is advanced into the first opening from the lower end of the body; and a load ring disposed in the second opening and axially secured relative to the body by slot-and-pin fasteners at respective, opposing locations on the arcuate walls and the load ring. The load ring has a pair of circumferentially spaced, upwardly oriented surfaces to define a saddle, the saddle configured to oppose portions of the head of the rod when the rod is inserted in the second opening.

The split ring has an inner surface defining an internal bowl, the internal bowl is slidably engageable with the head of the shank when received in the first opening to allow the head of the shank to be positioned in a user-selected one of a plurality of arcuate locations relative to the body of the tulip, thereby making the tulip polyaxial.

In another potential implementation, the body, split ring, and load ring comprise modular components. The split ring is securable within the first opening of the body by suitable rotation relative thereto. The load ring is securable after inserting into the second opening by means of pins received in slots defined on the load ring, thereby making the tulip modular.

In another potential implementation, the split ring has an associated height, inner, and outer diameters. The split ring has a split extending between the inner and outer diameters and through the entire height at a diagonal to define a diagonal cut. The split ring expands and contracts along the diagonal cut in response to the insertion of head of the shank therethrough.

In still further implementation, the first opening has a ring seat, and the split ring has a lower taper opposable against the ring seat, at least one of the ring seat and the lower taper having microserrations defined therein to resist unintended movement of the lower taper and seat relative to each other.

In one possible implementation, an assembly for a surgical implant procedure for the sacroiliac joint (SIJ) is provided. The assembly comprises a shank comprising three regions of varying porosity located between proximal and distal ends of the shank. The first region comprises a shank body having a longitudinal axis, head and a neck at the proximal end of the shank, and a core extending distally therefrom to terminate at a distal, insertion tip, the core having an outer core surface with bone engagement threads disposed thereon, an inner core surface defining a longitudinally extending chamber therein, and openings extending between the inner and outer core surfaces. The shank body comprises solid material selected and configured to support biomechanical loads on the shank when the implant extends to the pelvis. The second region comprises a strut-based microporous surface lattice extending longitudinally on the outer core surface and adapted to promote bone growth thereon. The third region comprises an open-sheet-based lattice extending longitudinally in the chamber within the core and accessible through the openings extending between the outer and inner core surfaces, the openings configured to receive bone matter into the chamber when bone matter is displaced during advancement of the shank into bone. The assembly further comprises a tulip for connecting to the shank and a rod associated with the surgical implant procedure for the sacroiliac joint (SIJ) is provided. The tulip comprises a cylindrical body having a longitudinal axis, and defined by a pair of arcuate walls extending longitudinally between upper and lower ends of the tulip, the arcuate walls extending circumferentially through respective arcs to define a pair of slots in the cylindrical body, the slots configured to be able to selectively receive the rod therethrough, the cylindrical body having first and second openings defined at the lower and upper ends of the body, respectively; a saddle disposed in the second opening. The saddle is axially secured relative to the body by a cam-and-cam-follower arrangement at respective, opposing locations on the arcuate walls and the saddle, the saddle having a pair of circumferentially spaced, upwardly oriented surfaces configured to oppose portions of the head of the rod when the rod is inserted in the second opening. The lower surface of the body terminates in a lower, body surface disposed at a first non-orthogonal angle relative to the longitudinal access corresponding to a favored angle associated with the SIJ procedure. The saddle has a lower, saddle surface also disposed to correspond to the favored angle.

In another possible implementation, a companion implant for the surgical implant procedure for the sacroiliac joint (SIJ) is provided. The implant is configured to be implantable in an implant trajectory of the SIJ procedure which is parallel to the fusion shank trajectory associated therewith. The companion implant comprises three regions of varying porosity located between proximal and distal ends of the implant. The first region comprises an implant body having a longitudinal axis, head and a neck at the proximal end of the implant, and a core extending distally therefrom to terminate at a distal, insertion tip, the core having an outer core surface with bone engagement threads disposed thereon, an inner core surface defining a longitudinally extending chamber therein, and openings extending between the inner and outer core surfaces. The body comprises solid material selected and configured to support biomechanical loads on the when the implant extends to the pelvis. The second region comprises a strut-based microporous surface lattice extending longitudinally on the outer core surface and adapted to promote bone growth thereon. The third region comprises an open-sheet-based lattice extending longitudinally in the chamber within the core and accessible through the openings extending between the outer and inner core surfaces, the openings configured to receive bone matter into the chamber when bone matter is displaced during advancement of the companion implant into bone.

The foregoing summary and this disclosure will be better understood with reference to the drawings.

### BRIEF DESCRIPTION OF DRAWINGS

A more complete understanding of the present disclosure, and the attendant advantages and features thereof, will be more readily understood by reference to the following detailed description, when considered in conjunction with the accompanying drawings, wherein:

FIG. 1 is an isometric view of one possible implementation of a polyaxial tulip for connecting to a shank and a rod associated with a spinal procedure according to the present disclosure;

FIG. 2 is an exploded, isometric view of the implementation of the polyaxial tulip assembly of FIG. 1, showing subcomponents according to the present disclosure;

FIGS. 3a and 3b are isometric, cut-away views of the implementation of the polyaxial tulip assembly of FIG. 1, showing subcomponents according to the present disclosure;

FIG. 4 is a cross-sectional view of the shank prior to insertion into the bottom of the polyaxial tulip according to the present disclosure;

FIG. 5 is a cross-sectional view showing the shank after insertion into the polyaxial tulip according to the present disclosure;

FIG. 6 is an isometric cross-sectional view of the polyaxial tulip, shank, rod, and lockscrew assembly;

FIGS. 7a and 7b are isometric views of one possible implementation of a shank for a surgical implant procedure for the sacroiliac joint (SIJ) according to the present disclosure;

FIGS. 8a and 8b are isometric views of one possible implementation of a lateral/companion implant according to the present disclosure; and

FIGS. 9a, 9b, and 9c are isometric views of a favored angle screw tulip for connecting to a shank and a rod associated with the surgical implant procedure for the sacroiliac joint (SIJ) according to the present disclosure.

### DETAILED DESCRIPTION

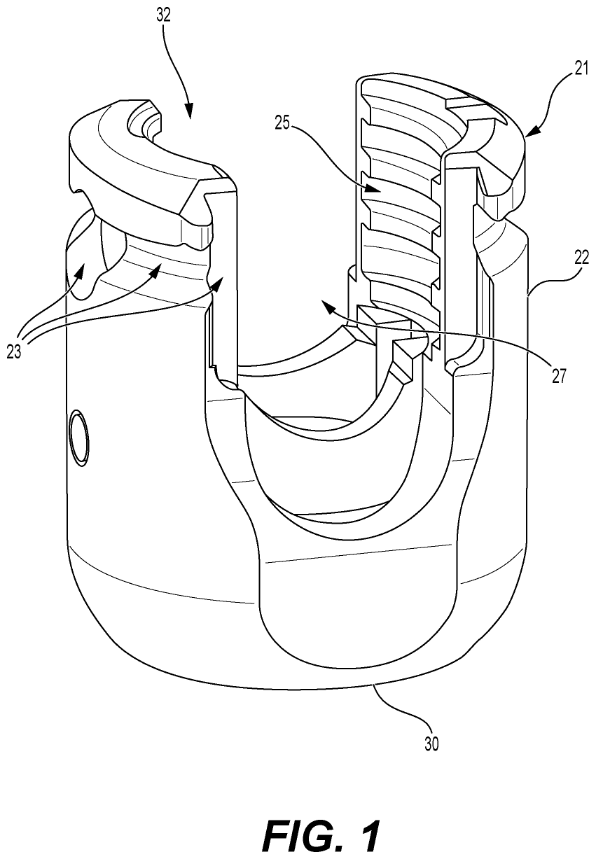

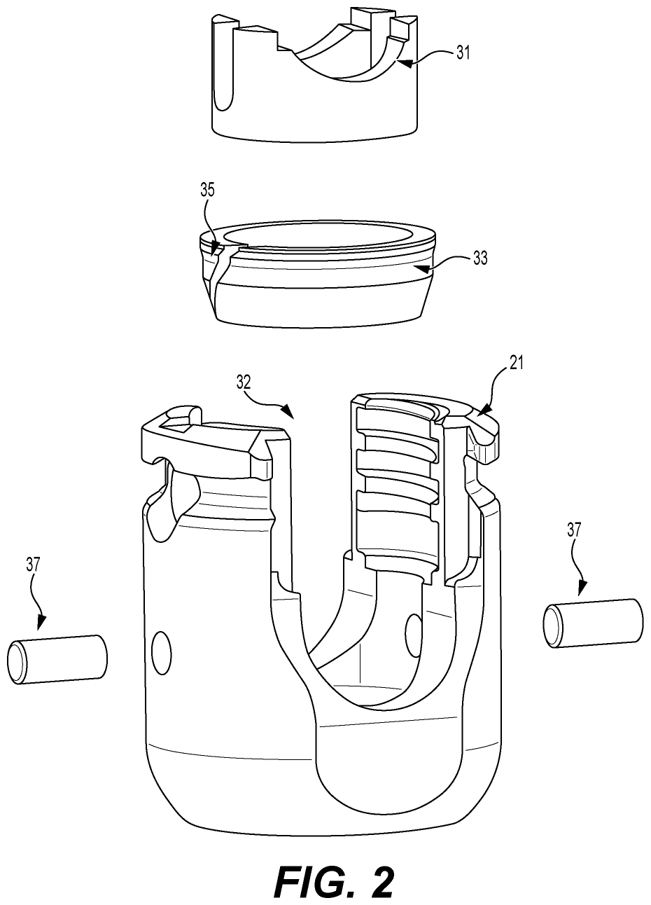

In one implementation, with this general background in mind, a polyaxial tulip is disclosed with reference to FIGS. 1-6.

Spinal fixation systems may include shank and tulip assemblies for associated spinal surgeries. Shank and tulip assemblies of the present disclosure may likewise include components which foster bone fusion, such as fusion of the sacroiliac joint, or a polyaxial tulip assembly or tulip.

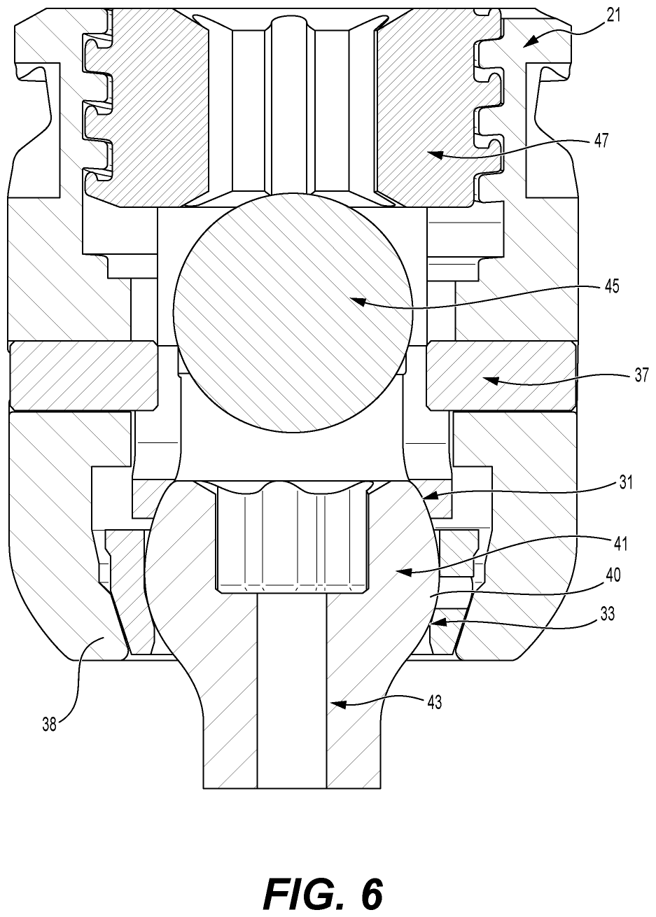

Tulip 21 comprises a cylindrical body 22 having a longitudinal axis and is defined by a pair of arcuate walls extending longitudinally between upper and lower ends of tulip 21. The arcuate walls of body 22 have outer surfaces defining instrument connection interfaces 23. The arcuate walls extend circumferentially through respective arcs to define a pair of slots 27 in the cylindrical body. The slots 27 are configured to be able to selectively receive a rod therethrough and lock down the rod into the tulip 21 by a lockscrew (shown later in FIG. 6). The cylindrical body has reverse buttress threads 25 defined therein and configured to engage the lockscrew.

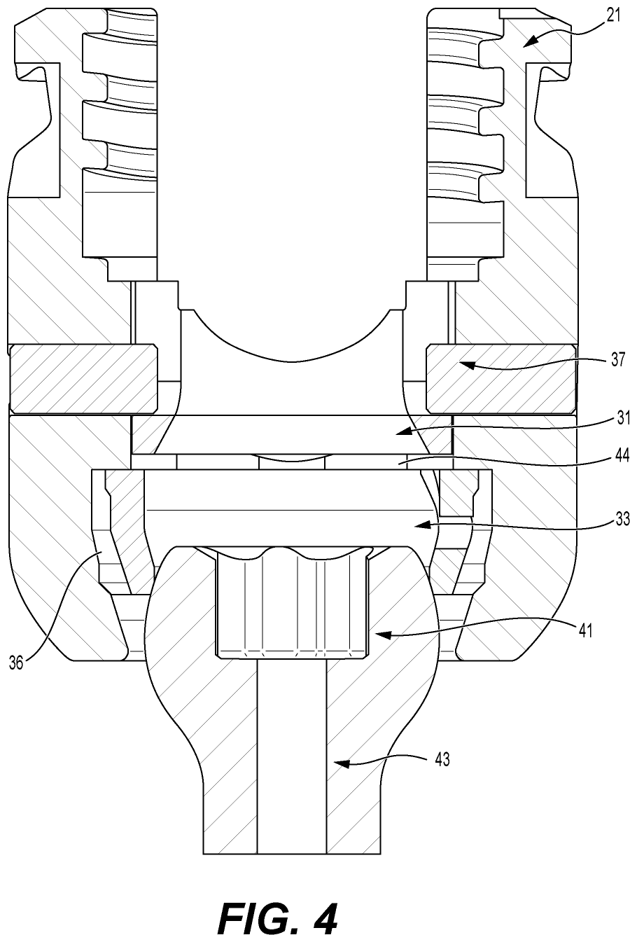

Polyaxial tulip 21 includes a load ring 31, a split ring 33 and pins 37. The cylindrical body 22 of the tulip 21 has first and second openings 30, 32 defined at the lower and upper ends of the body, respectively. The split ring 33 is disposed in the first opening 30, the split ring 33 having an inner diameter configured to secure a head of a shank when the shank is advanced into the first opening 30 from the lower end of the body (FIG. 6). Load ring 31 is disposed in the second opening 32 and axially secured relative to the body by slot-and-pin fasteners 37 at respective, opposing locations on the arcuate walls and the load ring 31. The load ring 31 has a pair of circumferentially spaced, upwardly oriented surfaces to define a saddle, the saddle configured to oppose portions of the head of the rod when the rod is inserted in the second opening. The split ring 33 has an inner surface defining an internal bowl 36, the internal bowl 36 is slidably engageable with the head of the shank (shown later in FIGS. 4-6) when received in the first opening to allow the head of the shank to be positioned in a user-selected one of a plurality of arcuate locations relative to the body of the tulip 21, thereby making the tulip 21 polyaxial.

The first opening has a ring seat 40 and the split ring 33 has a lower taper opposable against the ring seat, at least one of the ring seat and the lower taper having microserrations 38 defined therein to resist unintended movement of the lower taper and seat relative to each other. The microserrations reduces the incidence of tulip 21 disassociation from the shank during use.

The body 22, split ring 33, and load ring 31 comprise modular components. The split ring 33 is securable within the first opening of the body by suitable rotation relative thereto. The load ring 31 is securable after insertion into the second opening by means of pins 37 received in slots defined on the load ring 31, thereby making the tulip 21 modular.

The split ring 33 has an associated height, inner, and outer diameters. The split ring 33 has a split 35 extending between the inner and outer diameters and through the entire height at a diagonal to define a diagonal cut. The split ring 33 expands and contracts along the diagonal cut in response to the insertion of the head of the shank therethrough.

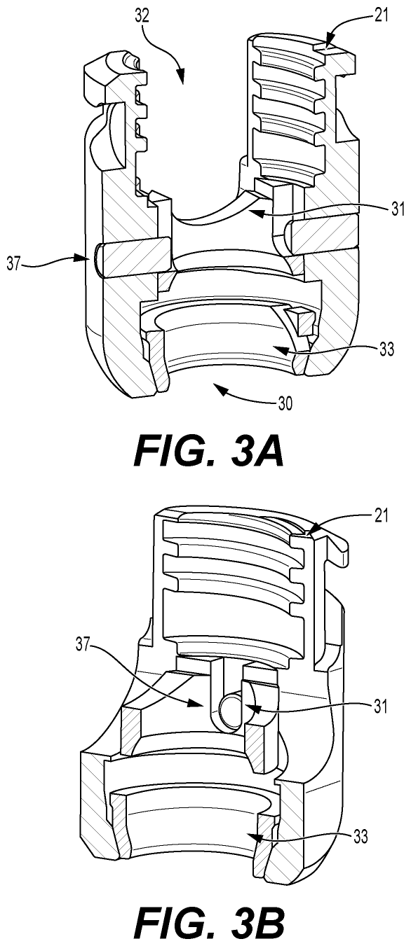

FIGS. 3a and 3b are isometric cut-away views of the polyaxial tulip 21, showing further aspects of the split ring 33, load ring 31, and pins 37. The split ring 33 is seen in the cut-away views of FIGS. 3a and 3b as disposed in the first opening, and the load ring 31 is likewise shown disposed in the second opening 32, the views further illustrating how load ring 31 is axially secured relative to the body by slot-and-pin fasteners at respective, opposing locations on the arcuate walls of the body 22 and load ring 31. The pins 37 may be laser welded to the tulip 21.

FIG. 4 is a cross-sectional view of polyaxial tulip 21 with shank 43. The split ring 33 disposed in the first opening 30, has an inner diameter configured to secure a head 41 of the shank 43 when the shank 43 is advanced into the first opening from the lower end of the body.

Body 22 in the illustrated implementation includes a flange 44 positioned to contact opposing surfaces of the load ring 31 and split ring 33, which helps resist tulip 21 disassociation from the rod when the load ring 31 is secured within body 22 by another instrument, such as lockscrew 47 (FIG. 6). The load ring 31 comprises a bottom surface defining a flared, opening facing into the first opening, the flared opening being configured to engage the head 41 of the shank 43 when received in the first opening.

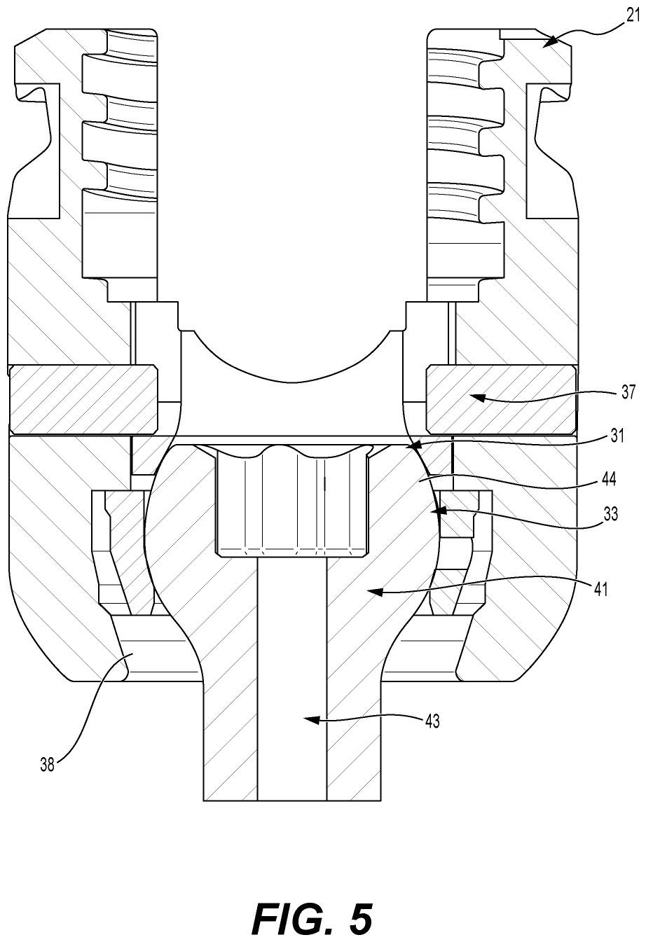

FIG. 5 is a cross-sectional view of the polyaxial tulip 21 showing the shank 43 secured thereto. The tulip 21 is configured to receive the shank 43 by means of the shank 43 being pushed upwardly against the split ring 33 until it reaches an opposing surface of load ring 31 flange 44 or a similar ceiling or stop structure in the tulip 21. During insertion the split ring 33 to expands until it is able to accept the shank head 41 through the bottom opening 30. Split ring 33 disposed in the first opening of the body is thus configured to secure the head 41 of the shank 43 when the shank 43 is advanced into the first opening from the lower end of the body. The spring nature of the split ring 33 allows the shank 43 to be inserted in one fluid motion.

The split ring 33 has an inner surface defining internal bowl 36. Internal bowl 36 is slidably engageable with the head 41 of the shank 43 when received in the first opening to allow the head 41 of the shank 43 to be positioned in a user-selected one of a plurality of arcuate locations relative to the body of the tulip 21. As such internal bowl into the split ring 33 gives the shank 43 the ability to rotate and be polyaxial with respect to the tulip 21, allowing a surgeon to use it in different clinical situations and with varying anatomy.

In a possible implementation, tulip body 22 consists of cobalt chrome alloy. The cobalt chrome alloy has a measured stiffness to reduce tulip splay and resist cross-threading of the lockscrew 47. The cobalt chrome alloy is stiffer than the titanium alloy. This reduces tulip splay and improves its resistance to cross threading of the lockscrew 47.

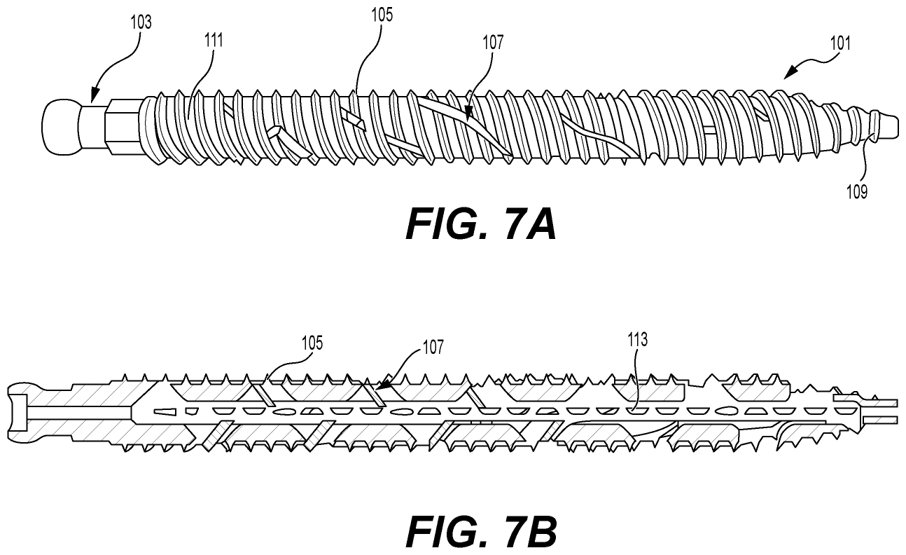

Spinal fixation systems of the present disclosure may likewise include components which foster bone fusion, such as fusion of the sacroiliac joint. In one possible implementation, an assembly for a surgical implant procedure for the sacroiliac joint (SIJ) is disclosed with reference to FIGS. 7a and 7b. The assembly comprises a shank 101 which comprises three regions of varying porosity located between proximal and distal ends of the shank 101. The first region comprises a shank body 103 having a longitudinal axis, head and a neck at the proximal end of the shank 101, and a core 107 extending distally therefrom to terminate at a distal, insertion tip 109, the core 107 having an outer core surface with bone engagement threads 105 disposed thereon, an inner core surface defining a longitudinally extending chamber therein, and openings extending between the inner and outer core surfaces. The shank body 103 comprises solid material selected and configured to support biomechanical loads on the shank 101 when the implant extends to the pelvis.

The second region comprises a strut-based microporous surface lattice 111 extending longitudinally on the outer core surface and adapted to promote bone growth thereon.

The third region comprises an open-sheet-based lattice 113 extending longitudinally in the chamber within the core 107 and accessible through the openings extending between the outer and inner core surfaces, the openings configured to receive bone matter into the chamber when bone matter is displaced during advancement of the shank 101 into bone.

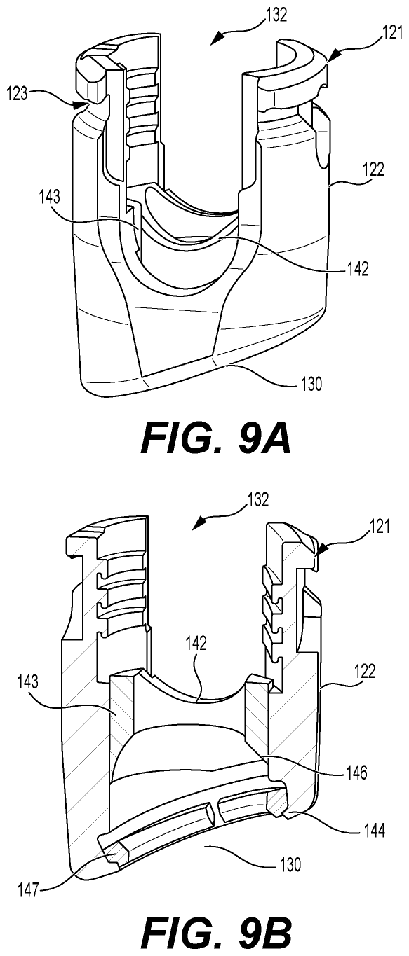

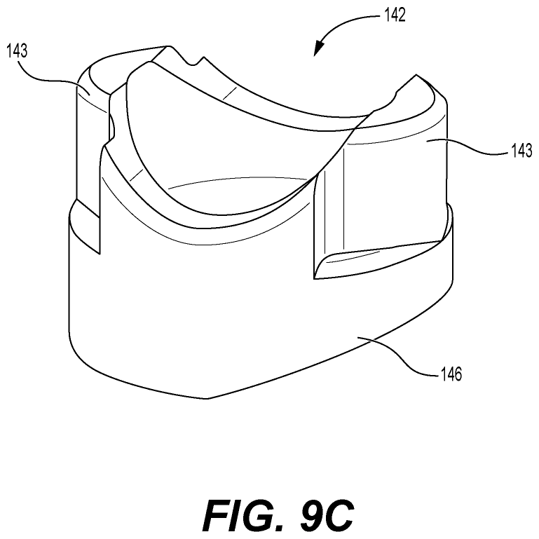

Furthermore, the assembly comprises a tulip 121 (as shown in FIGS. 9a, 9b, and 9c) for connecting to the shank 101 (or any other suitable shank) and a rod associated with the surgical implant procedure for the sacroiliac joint (SIJ). The tulip 121 comprises a cylindrical body 122 having a longitudinal axis, and defined by a pair of arcuate walls extending longitudinally between upper and lower ends of the tulip, the arcuate walls extending circumferentially through respective arcs to define a pair of slots in the cylindrical body 122, the slots configured to be able to selectively receive the rod therethrough, the cylindrical body 122 having first and second openings 130, 132 defined at the lower and upper ends of the body 122, respectively; a saddle 142 disposed in the second opening 132.

The saddle 142 is axially secured relative to the body 122 by a cam-and-cam-follower arrangement 143 at respective, opposing locations on the arcuate walls and the saddle, the saddle having a pair of circumferentially spaced, upwardly oriented surfaces configured to oppose portions of the head of the rod when the rod is inserted in the second opening 132. The lower surface of the body 122 terminates in a lower, body surface 144 disposed at a first non-orthogonal angle relative to the longitudinal axis corresponding to a favored angle associated with the SIJ procedure. The saddle 142 has a lower, saddle surface 146 also disposed to correspond to the favored angle.

The arcuate walls of the body 122 have outer surfaces defining instrument connection interfaces 123. The favored angle of the tulip 121 comprises 20 degrees. Furthermore, the first opening 130 is sized to receive the saddle therethrough. The tulip 121 further comprises a c-clip 147 loadable through the first opening 130 in the body 122 and securable relative thereto after receiving the saddle 142 and any suitable shank therein, including, by way of example, shank 101 of the present disclosure. The c-clip 147 includes an undercut and engages the inner surface of the body 122 in the first opening 130 to support the saddle 142 and maintain the saddle 142 secured relative to the body 122. The c-clip 147 is configured to define a 50-degree cone of angulation to the shank 101 when received in the body 122.

The shank 101 may be a monolithic, 3D printed porous shank for fusion of the Sacroiliac Joint (SIJ). The shank 101 utilizes three regions of varying porosity to achieve on-growth and in-growth while maintaining mechanical strength. The three separate regions provide a stable fixation while biologically integrating with the bone it is inserted into. The shank 101 has a spherical head that attaches to a favored angle tulip 121 in open, closed, and reduction configurations. A solid region at the head, neck, threads, and core of the body is used to provide mechanical strength to supporting the biomechanical loads of a construct that extends to the pelvis.

To support long construct deformity cases, this implant prioritizes strength first, with the additional benefit of osseointegration. At the spherical head, a Custom-Star 27 drive is used to deliver higher torque than legacy systems. At the neck, a 7 mm hex can be used as a bail-out engagement feature if the neck were to fail. A strut-based microporous surface lattice is used to promote on-growth, mitigating back-out of the screw, and promoting osseointegration. Deep flutes along the screw are designed to excavate and harvest bone. Additionally, these deep flutes reduce insertional torque and consequently help prevent coring and implant failure during insertion.

Along the center of the shank is an open sheet-based lattice. This lattice provides space for the harvested bone to enter for deep in-growth. This porous structure also reduces the structural elasticity of the implant, bringing it closer to that of bone and mitigating screw toggle.

The shank 101 is used in a posterior approach, and can be implanted on a variety of trajectories, including traditional S2AI, modified S2AI, traditional iliac, and modified iliac. The stronger neck of the shank 101 prevents the fractures compared to legacy product available in the market.

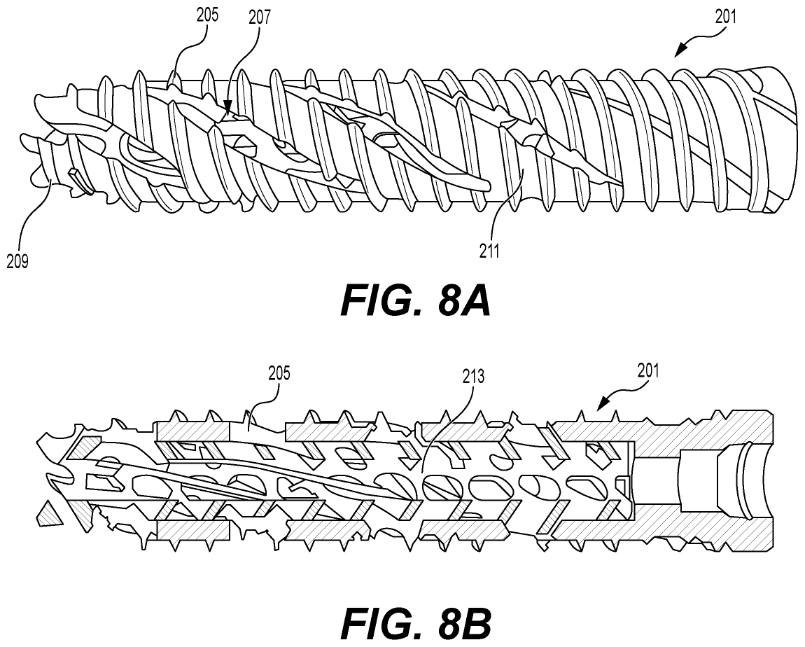

In another possible implementation, a companion/lateral implant 201 for the surgical implant procedure for the sacroiliac joint (SIJ) is disclosed with reference to FIGS. 8a and 8b. The companion implant 201 is configured to be implantable in an implant trajectory of the SIJ procedure which is parallel to the fusion shank 101 (shown in FIGS. 7a and 7b) trajectory associated therewith. The companion implant 201 comprises three regions of varying porosity located between proximal and distal ends of the implant 201.

The first region comprises an implant body having a longitudinal axis, head and a neck at the proximal end of the implant 201, and a core 207 extending distally therefrom to terminate at a distal, insertion tip 209, the core 207 having an outer core surface with bone engagement threads 205 disposed thereon, an inner core surface defining a longitudinally extending chamber therein, and openings extending between the inner and outer core surfaces. The body comprises solid material selected and configured to support biomechanical loads on the when the implant extends to the pelvis.

The second region comprises a strut-based microporous surface lattice 211 extending longitudinally on the outer core surface and adapted to promote bone growth thereon.

The third region comprises an open-sheet-based lattice 213 extending longitudinally in the chamber within the core 207 and accessible through the openings extending between the outer and inner core surfaces, the openings configured to receive bone matter into the chamber when bone matter is displaced during advancement of the companion implant 201 into bone.

The companion/lateral implant 201 is structured similarly to the S2AI, but with added emphasis on osseointegration. Osseointegration is crucial for long-term success because it ensures the implant becomes securely attached to the bone, preventing loosening or shifting. The companion/lateral implant 201 provides better self-tapping and harvesting performance.

The lateral implant 201 is configured to be implantable in an implant trajectory of the SIJ procedure which is parallel to the fusion shank trajectory associated therewith. Furthermore, the lateral implant 201 is configured for standalone use without the tulip 121 during an SIJ fusion procedure. The openings of the lateral implant 201 comprise flutes having a different pitch than the threads to receive bone matter displaced by adjacent threads.

The head of the lateral implant 201 is sized to deliver sufficient torque for SIJ procedures without surpassing the elastic limit of the implant or surpassing the biomechanical limits of the bone. Furthermore, the head comprises a custom-star 27 drive. The head also comprises a drive feature recessed into the proximal end of the implant, and wherein the head further comprises a circumferential channel cut configured to receive a corresponding clip ring.

It will be further understood that still further changes and variations in the details, materials, and arrangements of the components, parts, and features which have been described and illustrated may be made. All such modifications are deemed to be within the scope of this disclosure, as defined by the appended claims.

1. A polyaxial tulip for connecting to a shank and a rod associated with a spinal procedure, the tulip comprising:
a cylindrical body having a longitudinal axis, and defined by a pair of arcuate walls extending longitudinally between upper and lower ends of the tulip, the arcuate walls extending circumferentially through respective arcs to define a pair of slots in the cylindrical body, the slots configured to be able to selectively receive the rod therethrough, the cylindrical body having first and second openings defined at the lower and upper ends of the body, respectively;
a split ring disposed in the first opening, the split ring having an inner diameter configured to secure a head of the shank when the shank is advanced into the first opening from the lower end of the body; and
a load ring disposed in the second opening and axially secured relative to the body by slot-and-pin fasteners at respective, opposing locations on the arcuate walls and the load ring, the load ring having a pair of circumferentially spaced, upwardly oriented surfaces to define a saddle, the saddle configured to oppose portions of the rod when the rod is inserted in the second opening, wherein the load ring includes a pair of vertical elongate slots configured to receive the respective pins extending inwardly from the pair of arcuate walls and to slide vertically relative to the respective pins when the rod is being secured to the saddle,
wherein the split ring has an inner surface defining an internal bowl, and
wherein the internal bowl is slidably engageable with the head of the shank when received in the first opening to allow the head of the shank to be positioned in a user-selected one of a plurality of arcuate locations relative to the body of the tulip, thereby making the tulip polyaxial.

2. The tulip of claim 1, wherein the body, the split ring, and the load ring comprise modular components, the split ring securable within the first opening of the body by suitable rotation relative thereto, the load ring securable after insertion into the second opening by means of the pins received in the slots on the load ring, thereby making the tulip modular.

3. The tulip of claim 1, further comprising a lockscrew threadably receivable and advanceable in the second opening to secure positions of the rod and the shank when placed in the tulip, wherein each vertical elongate slot includes a closed bottom end and an open upper end such that the closed bottom ends prevent the load ring from moving upwardly when the head of the shank is being advanced into the first opening from the lower end of the body to be seated in the split ring and the load ring.

4. The tulip of claim 3, wherein the body consists of cobalt chrome alloy having a measured stiffness to reduce tulip splay and resist cross-threading of the lockscrew.

5. The tulip of claim 3, wherein the second opening has reverse buttress threads defined therein and configured to engage the lockscrew.

6. The tulip of claim 5 wherein the reverse buttress threads have a backdraft angle.

7. The tulip of claim 1, wherein the arcuate walls of the body have outer surfaces defining instrument connection interfaces.

8. The tulip of claim 1, wherein the split ring has an associated height, inner, and outer diameters, and wherein the split ring has a split extending between the inner and outer diameters and through an entirety of the associated height at a diagonal to define a diagonal cut, the split ring expanding and contracting along the diagonal cut in response to insertion of the head of the shank therethrough.

9. The tulip of claim 1, wherein the first opening has a ring seat and the split ring has a lower taper opposable against the ring seat, at least one of the ring seat and the lower taper having microserrations defined therein to resist unintended movement of the lower taper and seat relative to each other.

10. The tulip of claim 1, wherein the load ring comprises a bottom surface defining a flared opening facing into the first opening, the flared opening configured to engage the head of the shank when received in the first opening.

11. A polyaxial tulip for connecting to a shank and a rod associated with a spinal procedure, the tulip comprising:
a cylindrical body having a longitudinal axis, and defined by a pair of arcuate walls extending longitudinally between upper and lower ends of the tulip, the arcuate walls extending circumferentially through respective arcs to define a pair of slots in the cylindrical body, the slots configured to be able to selectively receive the rod therethrough, the cylindrical body having first and second openings defined at the lower and upper ends of the body, respectively;
a split ring disposed in the first opening, the split ring having an inner diameter configured to secure a head of the shank when the shank is advanced into the first opening from the lower end of the body; and
a load ring disposed in the second opening and axially secured relative to the body by slot-and-pin fasteners at respective, opposing locations on the arcuate walls and the load ring, the load ring having a pair of circumferentially spaced, upwardly oriented surfaces to define a saddle, the saddle configured to oppose portions of the rod when the rod is inserted in the second opening, wherein the load ring includes a pair of vertical elongate slots configured to receive the respective pins extending inwardly from the pair of arcuate walls and to slide vertically relative to the respective pins when the rod is being secured to the saddle;
wherein the split ring has an inner surface defining an internal bowl, the internal bowl is slidably engageable with the head of the shank when received in the first opening to allow the head of the shank to be positioned in a user-selected one of a plurality of arcuate locations relative to the body of the tulip, thereby making the tulip polyaxial;
wherein the tulip further comprises a lockscrew threadably receivable and advanceable in the second opening;
wherein the second opening has reverse buttress threads defined therein;
wherein the body, split ring, and load ring comprise modular components; and
wherein the first opening has a ring seat and the split ring has a lower taper opposable against the ring seat, at least one of the ring seat and the lower taper having microserrations defined therein.

12. The tulip of claim 11, wherein the split ring is securable within the first opening of the body by suitable rotation relative thereto, and the load ring is securable after insertion into the second opening by means of the pins received in the slots on the load ring.

13. The tulip of claim 11, wherein the lockscrew is advanceable in the second opening to secure positions of the rod and the shank when placed in the tulip, wherein each vertical elongate slot includes a closed bottom end and an open upper end such that the closed bottom ends prevent the load ring from moving upwardly when the head of the shank is being advanced into the first opening from the lower end of the body to be seated in the split ring and the load ring.

14. The tulip of claim 13, wherein the body consists of cobalt chrome alloy having a measured stiffness to reduce tulip splay and resist cross-threading of the lockscrew.

15. The tulip of claim 13, wherein the reverse buttress threads of the second opening are configured to engage the lockscrew.

16. The tulip of claim 15, wherein the reverse buttress threads have a backdraft angle.

17. The tulip of claim 11, wherein the arcuate walls of the body have outer surfaces defining instrument connection interfaces.

18. The tulip of claim 11, wherein the split ring has an associated height, inner, and outer diameters, and wherein the split ring has a split extending between the inner and outer diameters and through an entirety of the associated height at a diagonal to define a diagonal cut, the split ring expanding and contracting along the diagonal cut in response to insertion of head of the shank therethrough.

19. The tulip of claim 11, wherein the ring seat and the lower taper having the microserrations defined therein to resist unintended movement of the lower taper and seat relative to each other.

20. A polyaxial tulip for connecting to a shank and a rod associated with a spinal procedure, the tulip comprising:
a cylindrical body having a longitudinal axis, and defined by a pair of arcuate walls extending longitudinally between upper and lower ends of the tulip, the arcuate walls extending circumferentially through respective arcs to define a pair of slots in the cylindrical body, the slots configured to be able to selectively receive the rod therethrough, the cylindrical body having first and second openings defined at the lower and upper ends of the body, respectively;
a split ring disposed in the first opening, the split ring having an inner diameter configured to secure a head of the shank when the shank is advanced into the first opening from the lower end of the body; and
a load ring disposed in the second opening and axially secured relative to the body by slot-and-pin fasteners at respective, opposing locations on the arcuate walls and the load ring, the load ring having a pair of circumferentially spaced, upwardly oriented surfaces to define a saddle, the saddle configured to oppose portions of the head of the rod when the rod is inserted in the second opening, wherein the body includes a pair of vertical elongate slots configured to allow the respective pins to slide within the elongate slots when the rod is being secured to the saddle;
wherein the split ring has an inner surface defining an internal bowl, the internal bowl is slidably engageable with the head of the shank when received in the first opening to allow the head of the shank to be positioned in a user-selected one of a plurality of arcuate locations relative to the body of the tulip, thereby making the tulip polyaxial;
wherein the tulip further comprises a lockscrew threadably receivable and advanceable in the second opening;
wherein the second opening has reverse buttress threads defined therein;
wherein the body, split ring, and load ring comprise modular components;
wherein the first opening has a ring seat and the split ring has a lower taper opposable against the ring seat, at least one of the ring seat and the lower taper having microserrations defined therein; and
wherein the load ring comprises a bottom surface defining a flared, opening facing into the first opening, the flared opening configured to engage the head of the shank when received in the first opening.
